(19) United States
(12) Patent Application Publication
(10) Pub. No.: US 2018/0353624 A1
(43) Pub. Date: Dec. 13, 2018

(54) ADENO-ASSOCIATED VIRAL VECTORS USEFUL IN TREATMENT OF SPINAL MUSCULAR ATROPY

(71) Applicant: The Trustees of the University of Pennsylvania, Philadelphia, PA (US)

(72) Inventors: James M. Wilson, Philadelphia, PA (US); Christian Hinderer, Philadelphia, PA (US)

(21) Appl. No.: 16/061,109

(22) PCT Filed: Dec. 14, 2016
(86) PCT No.: PCT/US2016/066669

§ 371 Date: Jun. 11, 2018

Related U.S. Application Data
(60) Provisional application No. 62/267,012, filed on Dec. 14, 2015.

(51) Int. Cl. A61K 48/00; C07K 14/47; C12N 15/86; A61P 21/00; A61K 9/00; A61K 38/17

(52) U.S. Cl. CPC A61K 48/0066; C07K 14/4702; C12N 15/86; A61P 21/00; C12N 2810/6027; A61K 38/1709; C12N 2750/14143; C12N 2800/22; A61K 9/0085

(57) ABSTRACT

Compositions and methods useful in treating spinal muscular atrophy are provided. The compositions comprise a recombinant adeno-associated viral vector containing an AAV capsid, e.g., AAVrh.10 capsid, and nucleic acid sequences encoding a functional SMN protein. The methods involve administering these compositions to humans in need thereof.

### INCORPORATION-BY-REFERENCE OF MATERIAL SUBMITTED IN ELECTRONIC FORM

Applicant hereby incorporates by reference the Sequence Listing material filed in electronic form herewith. This file is labeled “16-7655PCT_SEQ_Listing_ST25.txt”.

### BACKGROUND OF THE INVENTION

Spinal muscular atrophy (SMA) is a neuromuscular disease caused by mutations in telomeric SMN1, a gene encoding a ubiquitously expressed protein (survival of motor neuron—SMN) involved in splicesome biogenesis. For unclear reasons SMN deficiency results in selective toxicity to lower motor neurons, resulting in progressive neuron loss and muscle weakness. The severity of the disease is modified by the copy number of a centromeric duplication of the homologous gene (SMN2), which carries a splice site mutation that results in production of only small amounts of the full length SMN transcript. Patients who carry 1-2 copies of SMN2 present with the severe form of SMA, characterized by onset in the first few months of life and rapid progression to respiratory failure. Patients with 3 copies of SMN2 generally exhibit an attenuated form of the disease, typically presenting after six months of age. Though many never gain the ability to walk, they rarely progress to respiratory failure, and often live into adulthood. Patients with four SMN2 copies may not present until adulthood with gradual onset of muscle weakness. There is no current treatment for SMA other than palliative care.

The correlation between loss of SMN function and disease severity makes SMA a potential target for gene therapy. Previous studies involving administration of an adeno-associated virus, AAV8-hSMN, to the CNS (central nervous system) in SMA-mouse models demonstrated expression of SMN in the spinal cord and that the SMA phenotype could be rescued; however, only modest preservation in the number of motor neurons was produced—and long term survival was not achieved. (Passini et al., 2010, J Clin Invest 120: 1253-1264).

The disease presents unique challenges for gene therapy, in part, because the SMN gene product is intracellular. Thus, robust transduction efficiency for the underlying subset of involved motor neurons is important for efficacy. An alternative approach to treatment studied the use of antisense oligonucleotides injected into the mouse CNS to redirect the splicing of SMN2 and boost production of SMN protein. (Passini et al. 2011, Sci Transl Med 3: 72ra18).

For gene therapy, AAV9 emerged as the vector of choice based on results achieved in animal studies involving the transfer of genes to the CNS. For example, based on dose-response studies of AAV9 transduction of SMN in SMA mouse models, Passini tested doses of AAV9 injected intrathecally in non-human primates (“NHPs”) to determine whether adequate transfer of a marker gene (Green Fluorescent Protein, “GFP”) to motor neurons could be achieved. (Passini et al., 2014, Human Gene Therapy 25:619-630). And others reported the widespread distribution of GFP in the CNS of mice and NHPs that received an intrathecal injection of AAV9. (Myer et al. 2014, Mol. Ther. 23:477-487 and Hinderer et al., 2014, Mol Ther 1: 14051). Systemic delivery of AAV9 has also been shown to cross the blood-brain barrier and achieve widespread gene transfer of GFP to the CNS. (Foust et al. 2009, Nature Biotech 27: 59-65; Duque et al. 2009, Mol Ther 17: 1187-1196).

Recently, an alternative AAV vector, AAVrh10, reported to be at least as efficient as AAV9 for transduction of many tissues in mice was analyzed to compare the ability to achieve gene transfer of the marker gene, GFP, to the CNS and PNS (peripheral nervous system) following intravascular delivery in neonatal mice. While low dose AAVrh10 appeared to induce higher transduction in the tissues tested, the differences were less evident at higher doses likely necessary for a therapeutic effect. (Tanguy, et al., 2015, Front Mol Neurosci 8: article 36).

What is needed are effective treatments for SMA.

### SUMMARY OF THE INVENTION

In one aspect, an adeno-associated viral vector (AAV) vector includes an AAVrh10 capsid and a vector genome which comprises AAV inverted terminal repeats (ITR(s)) and nucleic acid sequences encoding human survival of motor neuron (SMN) protein and expression control sequences that direct expression of the SMN in a host cell.

In a further aspect, the invention relates to a recombinant adeno-associated viral vector (rAAV) having an AAVrh10 capsid encasing a nucleic acid that contains an AAV ITR(s) (inverted terminal repeat) and encodes SMN controlled by a regulatory element(s) that directs SMN expression in host cells (“rAAV.SMN”) suitable for intrathecal administration to an animal subject. Such rAAV.SMNs are replication defective and advantageously can be used to deliver SMN to the CNS of subjects diagnosed with an SMN deficiency; particularly human subjects diagnosed with SMA. In a preferred embodiment, the rAAV transduces neurons in the brain and spinal cord, and particularly motor neurons. In another preferred embodiment, the rAAV of the invention is not neutralized by antisera to AAV9 capsid that may be present in the subject to be treated. In certain embodiments, the nucleic acid sequences encode SEQ ID NO: 1 or a sequence sharing at least 95% identity therewith.

In certain embodiment, the nucleic acid sequences encoding the human SMN protein (“hSMN”) protein can be codon-optimized. See, e.g, the nucleic acid sequence encoding the SMN protein is an SMN1 sequence of SEQ ID NO: 2, or a sequence sharing at least 70% identity therewith.

In another aspect, pharmaceutical compositions are provided which include a pharmaceutically acceptable carrier and an rAAV vector as described herein.

In yet another aspect, a method for treating spinal muscular atrophy in a subject is provided. The method includes administering a pharmaceutical composition as described herein to a subject in need thereof.

In yet another aspect, a method of expressing SMN in a subject is provided. In one embodiment, the method includes administering a pharmaceutical composition as described herein to a subject in need thereof.

Other aspects and advantages of the invention will be readily apparent from the following detailed description of the invention.

### BRIEF DESCRIPTION OF THE DRAWINGS

FIG. 1 is a diagram showing SMN vector genome structure. ITR=AAV2 inverted terminal repeat. CB7=chicken beta actin promoter with cytomegalovirus enhancer. RBG=rabbit beta globin polyadenylation signal.

FIG. 2 is a photomicrograph demonstrating human SMN expression in the spinal cord and dorsal root ganglion of a vector treated SMNΔ7 mouse. An expression construct consisting of a codon-optimized human SMN cDNA and CB promoter was packaged in an AAVrh10 capsid. 5×10¹⁰ GC were injected into the facial vein of newborn SMNΔ7 mice. The animals were sacrificed on postnatal day 17 and tissues stained with an antibody against human SMN (2B1, Santa Cruz). The spinal cord demonstrated occasional transduced cells, whereas the dorsal root ganglia were heavily transduced.

FIG. 3 is a Western blot of HEK 293 and Huh7 cell lysate+/−transfection with pAAV.CB7.CI.hSMN. Cells were transfected at 90% confluency with lipofectamine 2000 and harvested 48 hours later.

FIGS. 4A-4B are an alignment of native hSMN1, variant d (Accession no. NM_000344.3) (Subject; SEQ ID NO: 3) vs. the codon optimized sequence described herein (Query; SEQ ID NO: 2).

FIG. 5 is a plasmid map of an AAVrh.10.hSMN1 construct described herein.

FIG. 6 is a survival curve of SMNΔ7 pups treated IV with various doses of AAVrh.10.hSMN1 similar to what is described in Example 2.

### DETAILED DESCRIPTION OF THE INVENTION

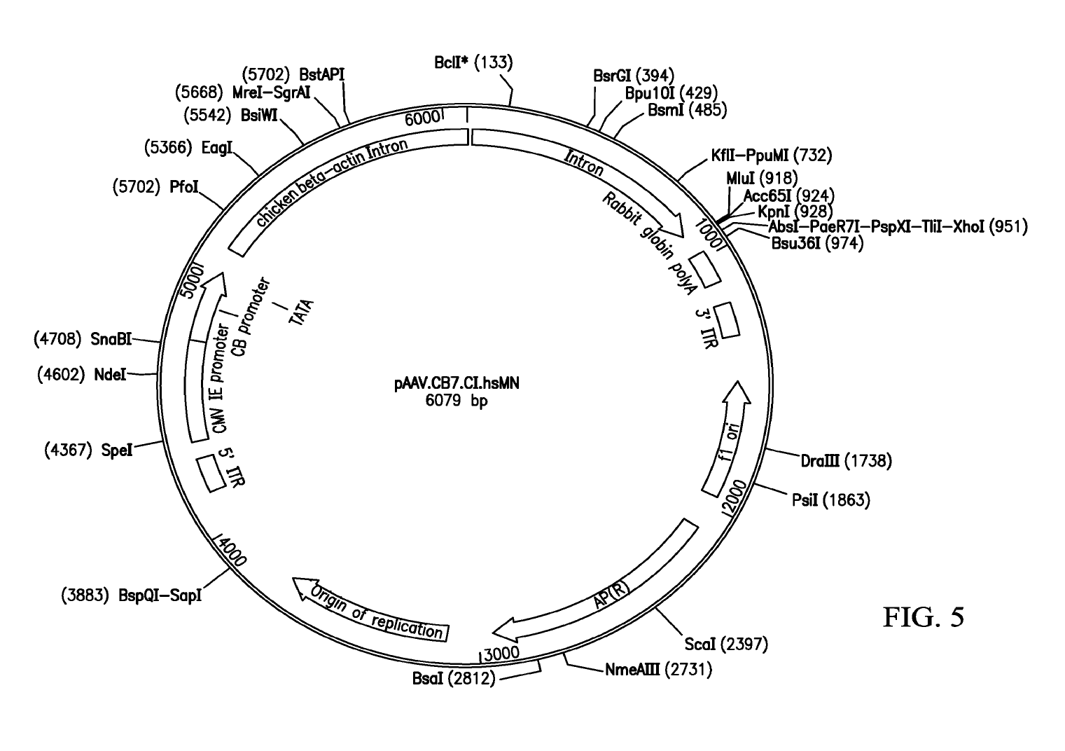

An engineered human (h) survival of motor neuron 1 (SMN1) cDNA is provided herein, which was designed to maximize translation as compared to the native hSMN1 sequence (as shown in FIG. 5, and SEQ ID NO: 3). An intron was incorporated upstream of the coding sequence to improve 5′ capping and stability of mRNA (see, FIG. 5 and SEQ ID NO: 4).

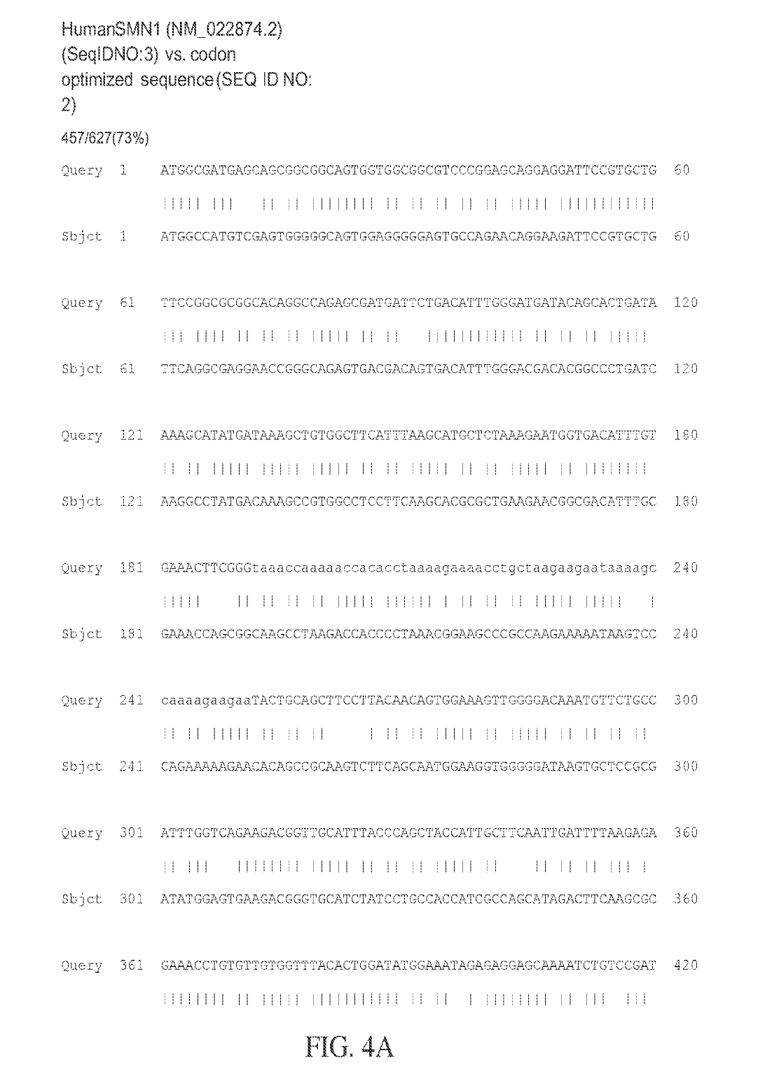

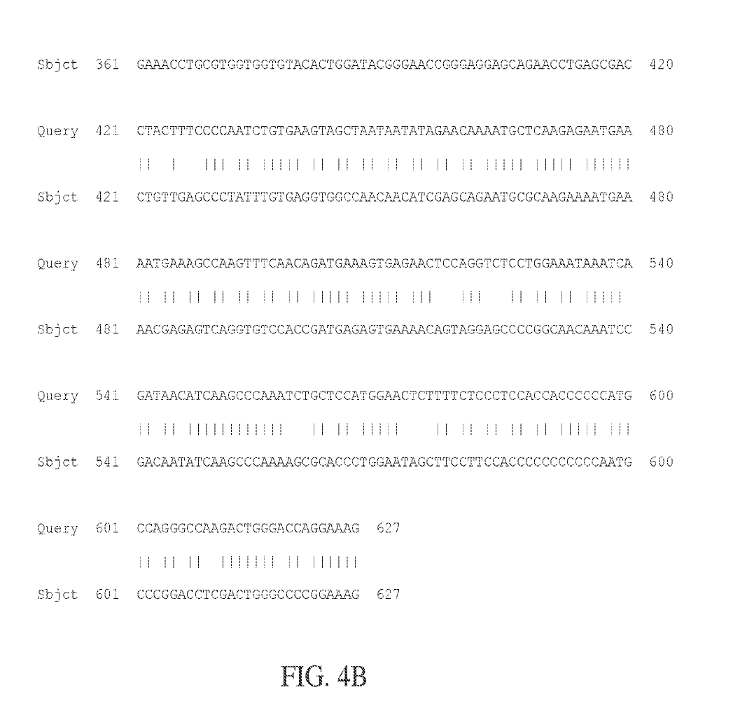

Also provided herein are viral vectors which include the engineered hSMN1 sequences. These compositions may be used in methods for the treatment of spinal muscular atrophy as described herein. For comparison purposes, an alignment of native human SMN1 coding sequence and an engineered cDNA is illustrated in FIG. 4.

The International SMA Consortium classification defines several degrees of severity in the SMA phenotype, depending on the age of onset and motor development milestones. SMA 0 designation is proposed to reflect prenatal onset and severe joint contractures, facial diplegia, and respiratory failure. Type I SMA, Werdnig-Hoffmann I disease, is the most severe post-natal form with onset within 6 months of birth. Patients are unable to sit up and have serious respiratory dysfunction. Type II SMA is the intermediate form with onset within the first 2 years; children can sit up but are unable to walk. The clinical course is variable. Type III (also called Kugelberg-Welander disease) begins after 2 years of age and usually has a chronic evolution. Children can stand and walk unaided at least in infancy. Adult form (type IV) is the mildest, with onset after 30 years of age; few cases have been reported and its prevalence is not accurately known.

SMA is an autosomal recessive disorder in which approximately 95% of SMA patients have homozygous absence of exons 7 and 8 (or exon 7 only) of the SMN1 gene. The remainder of patients are compound heterozygotes for SMN1 mutations, with a subtle mutation on one chromosome and a deletion or gene conversion on the other. Provision of a functioning SMN1 gene has been shown to rescue the phenotype. See, Tanguy, cited above.

In one aspect, a coding sequence is provided which encodes a functional SMN protein. In one embodiment, the amino acid sequence of the functional SMN1 is that of SEQ ID NO: 1 or a sequence sharing 95% identity therewith. In one embodiment, a modified hSMN1 coding sequence is provided. Preferably, the modified hSMN1 coding sequence has less than about 80% identity, preferably about 75% identity or less to the full-length native hSMN1 coding sequence (FIG. 4, SEQ ID NO: 3). In one embodiment, the modified hSMN1 coding sequence is characterized by improved translation rate as compared to native hSMN1 following AAV-mediated delivery (e.g., rAAV). In one embodiment, the modified hSMN1 coding sequence shares less than about 80%, 79%, 78%, 77%, 76%, 75%, 74%, 73%, 72%, 71%, 70%, 69%, 68%, 67%, 66%, 65%, 64%, 63%, 62%, 61% or less identity to the full length native hSMN1 coding sequence. In one embodiment, the modified hSMN1 coding sequence is SEQ ID NO: 2, or a sequence sharing 70%, 75%, 80%, 85%, 90%, 95% or greater identity with SEQ ID NO: 2.

The term “percent (%) identity”, “sequence identity”, “percent sequence identity”, or “percent identical” in the context of nucleic acid sequences refers to the residues in the two sequences which are the same when aligned for correspondence. The length of sequence identity comparison may be over the full-length of the genome, the full-length of a gene coding sequence, or a fragment of at least about 500 to 5000 nucleotides, is desired. However, identity among smaller fragments, e.g. of at least about nine nucleotides, usually at least about 20 to 24 nucleotides, at least about 28 to 32 nucleotides, at least about 36 or more nucleotides, may also be desired.

Percent identity may be readily determined for amino acid sequences over the full-length of a protein, polypeptide, about 32 amino acids, about 330 amino acids, or a peptide fragment thereof or the corresponding nucleic acid sequence coding sequences. A suitable amino acid fragment may be at least about 8 amino acids in length, and may be up to about 700 amino acids. Generally, when referring to “identity”, “homology”, or “similarity” between two different sequences, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence.

Alignments are performed using any of a variety of publicly or commercially available Multiple Sequence Alignment Programs. Sequence alignment programs are available for amino acid sequences, e.g., the “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

Multiple sequence alignment programs are also available for nucleic acid sequences. Examples of such programs include, “Clustal W”, “CAP Sequence Assembly”, “BLAST”, “MAP”, and “MEME”, which are accessible through Web Servers on the internet. Other sources for such programs are known to those of skill in the art. Alternatively, Vector NTI utilities are also used. There are also a number of algorithms known in the art that can be used to measure nucleotide sequence identity, including those contained in the programs described above. As another example, polynucleotide sequences can be compared using Fasta™, a program in GCG Version 6.1. Fasta™ provides alignments and percent sequence identity of the regions of the best overlap between the query and search sequences. For instance, percent sequence identity between nucleic acid sequences can be determined using Fasta™ with its default parameters (a word size of 6 and the NOPAM factor for the scoring matrix) as provided in GCG Version 6.1, herein incorporated by reference.

In one embodiment, the modified hSMN1 coding sequence is a codon optimized sequence, optimized for expression in the subject species. As used herein, the “subject” is a mammal, e.g., a human, mouse, rat, guinea pig, dog, cat, horse, cow, pig, or non-human primate, such as a monkey, chimpanzee, baboon or gorilla. In a preferred embodiment, the subject is a human. In one embodiment, the sequence is codon optimized for expression in a human.

Codon-optimized coding regions can be designed by various different methods. This optimization may be performed using methods which are available on-line (e.g., GeneArt), published methods, or a company which provides codon optimizing services, e.g., DNA2.0 (Menlo Park, Calif.). One codon optimizing method is described, e.g., in US International Patent Publication No. WO 2015/012924, which is incorporated by reference herein in its entirety. See also, e.g., US Patent Publication No. 2014/0032186 and US Patent Publication No. 2006/0136184. Suitably, the entire length of the open reading frame (ORF) for the product is modified. However, in some embodiments, only a fragment of the ORF may be altered. By using one of these methods, one can apply the frequencies to any given polypeptide sequence, and produce a nucleic acid fragment of a codon-optimized coding region which encodes the polypeptide.

A number of options are available for performing the actual changes to the codons or for synthesizing the codon-optimized coding regions designed as described herein. Such modifications or synthesis can be performed using standard and routine molecular biological manipulations well known to those of ordinary skill in the art. In one approach, a series of complementary oligonucleotide pairs of 80-90 nucleotides each in length and spanning the length of the desired sequence are synthesized by standard methods. These oligonucleotide pairs are synthesized such that upon annealing, they form double stranded fragments of 80-90 base pairs, containing cohesive ends, e.g., each oligonucleotide in the pair is synthesized to extend 3, 4, 5, 6, 7, 8, 9, 10, or more bases beyond the region that is complementary to the other oligonucleotide in the pair. The single-stranded ends of each pair of oligonucleotides are designed to anneal with the single-stranded end of another pair of oligonucleotides. The oligonucleotide pairs are allowed to anneal, and approximately five to six of these double-stranded fragments are then allowed to anneal together via the cohesive single stranded ends, and then they ligated together and cloned into a standard bacterial cloning vector, for example, a TOPO® vector available from Invitrogen Corporation, Carlsbad, Calif. The construct is then sequenced by standard methods. Several of these constructs consisting of 5 to 6 fragments of 80 to 90 base pair fragments ligated together, i.e., fragments of about 500 base pairs, are prepared, such that the entire desired sequence is represented in a series of plasmid constructs. The inserts of these plasmids are then cut with appropriate restriction enzymes and ligated together to form the final construct. The final construct is then cloned into a standard bacterial cloning vector, and sequenced. Additional methods would be immediately apparent to the skilled artisan. In addition, gene synthesis is readily available commercially.

In one embodiment, the modified hSMN1 genes described herein are engineered into a suitable genetic element (vector) useful for generating viral vectors and/or for delivery to a host cell, e.g., naked DNA, phage, transposon, cosmid, episome, etc., which transfers the hSMN1 sequences carried thereon. The selected vector may be delivered by any suitable method, including transfection, electroporation, liposome delivery, membrane fusion techniques, high velocity DNA-coated pellets, viral infection and protoplast fusion. The methods used to make such constructs are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y.

In one aspect, an expression cassette comprising the hSMN1 nucleic acid sequence(s) is provided. As used herein, an “expression cassette” refers to a nucleic acid molecule which comprises the hSMN1 sequence, promoter, and may include other regulatory sequences therefor, which cassette may be packaged into the capsid of a viral vector (e.g., a viral particle). Typically, such an expression cassette for generating a viral vector contains the hSMN1 sequence described herein flanked by packaging signals of the viral genome and other expression control sequences such as those described herein. For example, for an AAV viral vector, the packaging signals are the 5′ inverted terminal repeat (ITR) and the 3′ ITR. When packaged into the AAV capsid, the ITRs in conjunction with the expression cassette, are referred to herein as the “recombinant AAV (rAAV) genome” or “vector genome”.

Thus, in one aspect, an adeno-associated viral vector is provided which comprises an AAV capsid and at least one expression cassette, wherein the at least one expression cassette comprises nucleic acid sequences encoding SMN1 and expression control sequences that direct expression of the SMN1 sequences in a host cell. The AAV vector also comprises AAV ITR sequences. In one embodiment, the ITRs are from an AAV different than that supplying a capsid. In a preferred embodiment, the ITR sequences are from AAV2, or the deleted version thereof (ΔITR), which may be used for convenience and to accelerate regulatory approval. However, ITRs from other AAV sources may be selected. Where the source of the ITRs is from AAV2 and the AAV capsid is from another AAV source, the resulting vector may be termed pseudotyped. Typically, AAV vector genome comprises an AAV 5′ ITR, the hSMN1 coding sequences and any regulatory sequences, and an AAV 3′ ITR. However, other configurations of these elements may be suitable. A shortened version of the 5′ ITR, termed ΔITR, has been described in which the D-sequence and terminal resolution site (trs) are deleted. In other embodiments, the full-length AAV 5′ and 3′ ITRs are used.

In one aspect, a construct is provided which is a DNA molecule (e.g., a plasmid) useful for generating viral vectors. An illustrative plasmid containing desirable vector elements is illustrated by pAAV.CB7.CI.hSMN, a map of which is shown in FIG. 5, and the sequence of which is SEQ ID NO: 4, which is incorporated by reference. This illustrative plasmid contains an nucleic acid sequences comprising: 5′ ITR (nt 4150-4279 of SEQ ID NO: 4), a TATA signal (nt 4985-4988 of SEQ ID NO: 4), a synthetic hSMN1 coding sequence (nt 18-899 of SEQ ID NO: 4), a poly A (nt 984-1110 of SEQ ID NO: 4), a 3′ ITR (nt 1199-1328 of SEQ ID NO: 4), a CMV enhancer (nt 4347-4728 of SEQ ID NO: 4) a chicken beta-actin intron (nt 5107-6079 of SEQ ID NO: 4) and a CB promoter (nt 4731-5012 of SEQ ID NO: 4). Other expression cassettes may be generated using other synthetic hSMN1 coding sequences as described herein, and other expression control elements, described herein.

The expression cassette typically contains a promoter sequence as part of the expression control sequences, e.g., located between the selected 5′ ITR sequence and the hSMN1 coding sequence. The illustrative plasmid and vector described herein uses the ubiquitous chicken β-actin promoter (CB) with CMV immediate early enhancer (CMV IE). Alternatively, other neuron-specific promoters may be used [see, e.g., the Lockery Lab neuron-specific promoters database, accessed at http://chinook.uoregon.edu/promoters.html]. Such neuron-specific promoters include, without limitation, e.g., synapsin I (SYN), calcium/calmodulin-dependent protein kinase II, tubulin alpha I, neuron-specific enolase and platelet-derived growth factor beta chain promoters. See, Hioki et al, Gene Therapy, June 2007, 14(11):872-82, which is incorporated herein by reference. Other neuron-specific promoters include the 67 kDa glutamic acid decarboxylase (GAD67), homeobox Dlx5/6, glutamate receptor 1 (GluR1), preprotachykinin 1 (Tac1) promoter, neuron-specific enolase (NSE) and dopaminergic receptor 1 (Drd1a) promoters. See, e.g., Delzor et al, Human Gene Therapy Methods. August 2012, 23(4): 242-254. In another embodiment, the promoter is a GUSb promoter http://www.jci.org/articles/view/41615#B30.

Other promoters, such as constitutive promoters, regulatable promoters [see, e.g., WO 2011/126808 and WO 2013/04943], or a promoter responsive to physiologic cues may be used may be utilized in the vectors described herein. The promoter(s) can be selected from different sources, e.g., human cytomegalovirus (CMV) immediate-early enhancer/promoter, the SV40 early enhancer/promoter, the JC polymovirus promoter, myelin basic protein (MBP) or glial fibrillary acidic protein (GFAP) promoters, herpes simplex virus (HSV-1) latency associated promoter (LAP), rouse sarcoma virus (RSV) long terminal repeat (LTR) promoter, neuron-specific promoter (NSE), platelet derived growth factor (PDGF) promoter, hSYN, melanin-concentrating hormone (MCH) promoter, CBA, matrix metalloprotein promoter (MPP), and the chicken beta-actin promoter.

In addition to a promoter, an expression cassette and/or a vector may contain one or more other appropriate transcription initiation, termination, enhancer sequences, efficient RNA processing signals such as splicing and polyadenylation (polyA) signals; sequences that stabilize cytoplasmic mRNA for example WPRE; sequences that enhance translation efficiency (i.e., Kozak consensus sequence); sequences that enhance protein stability; and when desired, sequences that enhance secretion of the encoded product. Examples of suitable polyA sequences include, e.g., SV40, SV50, bovine growth hormone (bGH), human growth hormone, and synthetic polyAs. An example of a suitable enhancer is the CMV enhancer. Other suitable enhancers include those that are appropriate for CNS indications. In one embodiment, the expression cassette comprises one or more expression enhancers. In one embodiment, the expression cassette contains two or more expression enhancers. These enhancers may be the same or may differ from one another. For example, an enhancer may include a CMV immediate early enhancer. This enhancer may be present in two copies which are located adjacent to one another. Alternatively, the dual copies of the enhancer may be separated by one or more sequences. In still another embodiment, the expression cassette further contains an intron, e.g, the chicken beta-actin intron. Other suitable introns include those known in the art, e.g., such as are described in WO 2011/126808. Optionally, one or more sequences may be selected to stabilize mRNA. An example of such a sequence is a modified WPRE sequence, which may be engineered upstream of the polyA sequence and downstream of the coding sequence [see, e.g., MA Zanta-Boussif, et al, Gene Therapy (2009) 16: 605-619.

These control sequences are “operably linked” to the hSMN1 gene sequences. As used herein, the term “operably linked” refers to both expression control sequences that are contiguous with the gene of interest and expression control sequences that act in trans or at a distance to control the gene of interest.

An adeno-associated virus (AAV) viral vector is an AAV DNase-resistant particle having an AAV protein capsid into which is packaged nucleic acid sequences for delivery to target cells. An AAV capsid is composed of 60 capsid (cap) protein subunits, VP1, VP2, and VP3, that are arranged in an icosahedral symmetry in a ratio of approximately 1:1:10 to 1:1:20, depending upon the selected AAV. The AAV capsid may be chosen from those known in the art, including variants thereof. In one embodiment, the AAV capsid is chosen from those that effectively transduce neuronal cells. In one embodiment, the AAV capsid is selected from AAV1, AAV2, AAV7, AAV8, AAV9, AAVrh.10, AAV5, AAVhu.11, AAV8DJ, AAVhu.32, AAVhu.37, AAVpi.2, AAVrh.8, AAVhu.48R3 and variants thereof. See, Royo, et al, Brain Res, 2008 January, 1190:15-22; Petrosyan et al, Gene Therapy, 2014 December, 21(12):991-1000; Holehonnur et al, BMC Neuroscience, 2014, 15:28; and Cearley et al, Mol Ther. 2008 October; 16(10): 1710-1718, each of which is incorporated herein by reference. Other AAV capsids useful herein include AAVrh.39, AAVrh.20, AAVrh.25, AAV10, AAVbb.1, and AAV bb.2 and variants thereof. Other AAV serotypes may be selected as sources for capsids of AAV viral vectors (DNase resistant viral particles) including, e.g., AAV1, AAV2, AAV3, AAV4, AAV5, AAV6, AAV6.2, AAV7, AAV8, AAV9, rh10, AAVrh64R1, AAVrh64R2, rh8, rh.10, variants of any of the known or mentioned AAVs or AAVs yet to be discovered. See, e.g., US Published Patent Application No. 2007-0036760-A1; US Published Patent Application No. 2009-0197338-A1; EP 1310571. See also, WO 2003/042397 (AAV7 and other simian AAV), U.S. Pat. No. 7,790,449 and U.S. Pat. No. 7,282,199 (AAV8), WO 2005/033321 and U.S. Pat. No. 7,906,111 (AAV9), and WO 2006/110689, and WO 2003/042397 (rh.10). Alternatively, a recombinant AAV based upon any of the recited AAVs, may be used as a source for the AAV capsid. These documents also describe other AAV which may be selected for generating AAV and are incorporated by reference. In some embodiments, an AAV cap for use in the viral vector can be generated by mutagenesis (i.e., by insertions, deletions, or substitutions) of one of the aforementioned AAV Caps or its encoding nucleic acid. In some embodiments, the AAV capsid is chimeric, comprising domains from two or three or four or more of the aforementioned AAV capsid proteins. In some embodiments, the AAV capsid is a mosaic of Vp1, Vp2, and Vp3 monomers from two or three different AAVs or recombinant AAVs. In some embodiments, an rAAV composition comprises more than one of the aforementioned Caps. As used herein, relating to AAV, the term variant means any AAV sequence which is derived from a known AAV sequence, including those sharing at least 70%, at least 75%, at least 80%, at least 85%, at least 90%, at least 95%, at least 97%, at least 99% or greater sequence identity over the amino acid or nucleic acid sequence. In another embodiment, the AAV capsid includes variants which may include up to about 10% variation from any described or known AAV capsid sequence. That is, the AAV capsid shares about 90% identity to about 99.9% identity, about 95% to about 99% identity or about 97% to about 98% identity to an AAV capsid provided herein and/or known in the art. In one embodiment, the AAV capsid shares at least 95% identity with an AAV capsid. When determining the percent identity of an AAV capsid, the comparison may be made over any of the variable proteins (e.g., vp1, vp2, or vp3). In one embodiment, the AAV capsid shares at least 95% identity with the AAV8 vp3. In another embodiment, a self-complementary AAV is used.

In one embodiment, the capsid is an AAVrh.10 capsid, or a variant thereof. As used herein, “AAVrh10 capsid” refers to the rh.10 having the amino acid sequence of GenBank, accession: AA088201, which is incorporated by reference herein. This sequence is also reproduced in SEQ ID NO: 5. Some variation from this encoded sequence is acceptable, which may include sequences having about 99% identity to the referenced amino acid sequence in SEQ ID NO: 5, AA088201 and US 2013/0045186A1. Methods of generating the capsid, coding sequences therefore, and methods for production of rAAV viral vectors have been described. See, e.g., Gao, et al, Proc. Natl. Acad. Sci. U.S.A. 100 (10), 6081-6086 (2003) and US 2013/0045186A1. Other capsids, such as, e.g., those described in WO 2003/042397; WO 2005/033321, WO 2006/110689; U.S. Pat. No. 7,588,772 B2, which are incorporated by reference herein may be used in human subjects.

In one embodiment, a self-complementary AAV is provided. The abbreviation “sc” in this context refers to self-complementary. “Self-complementary AAV” refers a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety.

Methods for generating and isolating AAV viral vectors suitable for delivery to a subject are known in the art. See, e.g. US Published Patent Application No. 2007/0036760 (Feb. 15, 2007), U.S. Pat. No. 7,790,449; U.S. Pat. No. 7,282,199; WO 2003/042397; WO 2005/033321, WO 2006/110689; and U.S. Pat. No. 7,588,772 B2]. In a one system, a producer cell line is transiently transfected with a construct that encodes the transgene flanked by ITRs and a construct(s) that encodes rep and cap. In a second system, a packaging cell line that stably supplies rep and cap is transiently transfected with a construct encoding the transgene flanked by ITRs. In each of these systems, AAV virions are produced in response to infection with helper adenovirus or herpesvirus, requiring the separation of the rAAVs from contaminating virus. More recently, systems have been developed that do not require infection with helper virus to recover the AAV—the required helper functions (i.e., adenovirus E1, E2a, VA, and E4 or herpesvirus UL5, UL8, UL52, and UL29, and herpesvirus polymerase) are also supplied, in trans, by the system. In these newer systems, the helper functions can be supplied by transient transfection of the cells with constructs that encode the required helper functions, or the cells can be engineered to stably contain genes encoding the helper functions, the expression of which can be controlled at the transcriptional or posttranscriptional level. In yet another system, the transgene flanked by ITRs and rep/cap genes are introduced into insect cells by infection with baculovirus-based vectors. For reviews on these production systems, see generally, e.g., Zhang et al., 2009, “Adenovirus-adeno-associated virus hybrid for large-scale recombinant adeno-associated virus production,” Human Gene Therapy 20:922-929, the contents of each of which is incorporated herein by reference in its entirety. Methods of making and using these and other AAV production systems are also described in the following U.S. patents, the contents of each of which is incorporated herein by reference in its entirety: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065.

Optionally, the hSMN1 genes described herein may be used to generate viral vectors other than rAAV. Such other viral vectors may include any virus suitable for gene therapy may be used, including but not limited to adenovirus; herpes virus; lentivirus; retrovirus; etc. Suitably, where one of these other vectors is generated, it is produced as a replication-defective viral vector.

A “replication-defective virus” or “viral vector” refers to a synthetic or artificial viral particle in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless”-containing only the transgene of interest flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication. Such replication-defective viruses may be adeno-associated viruses (AAV), adenoviruses, lentiviruses (integrating or non-integrating), or another suitable virus source.

Also provided herein are pharmaceutical compositions. The pharmaceutical compositions described herein are designed for delivery to subjects in need thereof by any suitable route or a combination of different routes. In one embodiment, direct delivery to the CNS is desired and may be performed via intrathecal injection. The term “intrathecal administration” refers to delivery that targets the cerebrospinal fluid (CSF). This may be done by direct injection into the ventricular or lumbar CSF, by suboccipital puncture, or by other suitable means. Meyer et al, Molecular Therapy (31 Oct. 2014), demonstrated the efficacy of direct CSF injection which resulted in widespread transgene expression throughout the spinal cord in mice and nonhuman primates when using a 10 times lower dose compared to the IV application. This document is incorporated herein by reference. In one embodiment, the composition is delivered via intracerebroventricular viral injection (see, e.g., Kim et al, J Vis Exp. 2014 Sep. 15; (91):51863, which is incorporated herein by reference). See also, Passini et al, Hum Gene Ther. 2014 July; 25(7):619-30, which is incorporated herein by reference. In another embodiment, the composition is delivered via lumbar injection.

Typically, these delivery means are designed to avoid direct systemic delivery of the suspension containing the AAV composition(s) described herein. Suitably, this may have the benefit of reducing dose as compared to systemic administration, reducing toxicity and/or reducing undesirable immune responses to the AAV and/or transgene product.

Alternatively, other routes of administration may be selected (e.g., oral, inhalation, intranasal, intratracheal, intraarterial, intraocular, intravenous, intramuscular, and other parental routes).

The hSMN1 delivery constructs described herein may be delivered in a single composition or multiple compositions. Optionally, two or more different AAV may be delivered [see, e.g., WO 2011/126808 and WO 2013/049493]. In another embodiment, such multiple viruses may contain different replication-defective viruses (e.g., AAV, adenovirus, and/or lentivirus). Alternatively, delivery may be mediated by non-viral constructs, e.g., “naked DNA”, “naked plasmid DNA”, RNA, and mRNA; coupled with various delivery compositions and nano particles, including, e.g., micelles, liposomes, cationic lipid-nucleic acid compositions, poly-glycan compositions and other polymers, lipid and/or cholesterol-based-nucleic acid conjugates, and other constructs such as are described herein. See, e.g., X. Su et al, Mol. Pharmaceutics, 2011, 8 (3), pp 774-787; web publication: Mar. 21, 2011; WO2013/182683, WO 2010/053572 and WO 2012/170930, both of which are incorporated herein by reference, Such non-viral hSMN1 delivery constructs may be administered by the routes described previously.

The viral vectors, or non-viral DNA or RNA transfer moieties, can be formulated with a physiologically acceptable carrier for use in gene transfer and gene therapy applications. A number of suitable purification methods may be selected. Examples of suitable purification methods for separating empty capsids from vector particles are described, e.g., the process described in International Patent Application No. PCT/US16/65976, filed Dec. 9, 2016 and its priority documents US Patent Application Nos. 62/322,098, filed Apr. 13, 2016 and U.S. Patent Appln No. 62/266,341, filed on Dec. 11, 2015, and entitled “Scalable Purification Method for AAV8”, which is incorporated by reference herein. See, also, purification methods described in International Patent Application No. PCT/US16/65974, filed Dec. 9, 2016, and its priority documents, U.S. Patent Applications No. 62/322,083, filed Apr. 13, 2016 and 62/266,351, filed Dec. 11, 2015 (AAV1); International Patent Appln No. PCT/US16/66013, filed Dec. 9, 2016 and its priority documents U.S. Provisional Applications No. 62/322,055, filed Apr. 13, 2016 and 62/266,347, filed Dec. 11, 2015 (AAVrh10); and International Patent Application No. PCT/US16/65970, filed Dec. 9, 2016, and its priority applications U.S. Provisional Application Nos. 62/266,357 and 62/266,357 (AAV9), which are incorporated by reference herein. Briefly, a two-step purification scheme is described which selectively captures and isolates the genome-containing rAAV vector particles from the clarified, concentrated supernatant of a rAAV production cell culture. The process utilizes an affinity capture method performed at a high salt concentration followed by an anion exchange resin method performed at high pH to provide rAAV vector particles which are substantially free of rAAV intermediates.

In the case of AAV viral vectors, quantification of the genome copies (“GC”) may be used as the measure of the dose contained in the formulation. Any method known in the art can be used to determine the genome copy (GC) number of the replication-defective virus compositions of the invention. One method for performing AAV GC number titration is as follows: Purified AAV vector samples are first treated with DNase to eliminate contaminating host DNA from the production process. The DNase resistant particles are then subjected to heat treatment to release the genome from the capsid. The released genomes are then quantitated by real-time PCR using primer/probe sets targeting specific region of the viral genome (for example poly A signal). Another suitable method for determining genome copies are the quantitative-PCR (qPCR), particularly the optimized qPCR or digital droplet PCR [Lock Martin, et al, Human Gene Therapy Methods. April 2014, 25(2): 115-125. doi:10.1089/hgtb.2013.131, published online ahead of editing Dec. 13, 2013].

The replication-defective virus compositions can be formulated in dosage units to contain an amount of replication-defective virus that is in the range of about 1.0×10⁹ GC to about 1.0×10¹⁵ GC (to treat an average subject of 70 kg in body weight) including all integers or fractional amounts within the range, and preferably 1.0×10¹² GC to 1.0×10¹⁴ GC for a human patient. In one embodiment, the compositions are formulated to contain at least 1×10⁹, 2×10⁹, 3×10⁹, 4×10⁹, 5×10⁹, 6×10⁹, 7×10⁹, 8×10⁹, or 9×10⁹ GC per dose including all integers or fractional amounts within the range. In another embodiment, the compositions are formulated to contain at least 1×10¹⁰, 2×10¹⁰, 3×10¹⁰, 4×10¹⁰, 5×10¹⁰, 6×10¹⁰, 7×10¹⁰, 8×10¹⁰, or 9×10¹⁰ GC per dose including all integers or fractional amounts within the range. In another embodiment, the compositions are formulated to contain at least 1×10¹¹, 2×10¹¹, 3×10¹¹, 4×10¹¹, 5×10¹¹, 6×10¹¹, 7×10¹¹, 8×10¹¹, or 9×10¹¹ GC per dose including all integers or fractional amounts within the range. In another embodiment, the compositions are formulated to contain at least 1×10¹², 2×10¹², 3×10¹², 4×10¹², 5×10¹², 6×10¹², 7×10¹², 8×10¹², or 9×10¹² GC per dose including all integers or fractional amounts within the range. In another embodiment, the compositions are formulated to contain at least 1×10¹³, 2×10¹³, 3×10¹³, 4×10¹³, 5×10¹³, 6×10¹³, 7×10¹³, 8×10¹³,or 9×10¹³ GC per dose including all integers or fractional amounts within the range. In another embodiment, the compositions are formulated to contain at least 1×10¹⁴, 2×10¹⁴, 3×10¹⁴, 4×10¹⁴, 5×10¹⁴, 6×10¹⁴, 7×10¹⁴, 8×10¹⁴, or 9×10¹⁴ GC per dose including all integers or fractional amounts within the range. In another embodiment, the compositions are formulated to contain at least 1×10¹⁵, 2×10¹⁵, 3×10¹⁵, 4×10¹⁵, 5×10¹⁵, 6×10¹⁵, 7×10¹⁵, 8×10¹⁵, or 9×10¹⁵ GC per dose including all integers or fractional amounts within the range. In one embodiment, for human application the dose can range from 1×10¹⁰ to about 1×10¹² GC per dose including all integers or fractional amounts within the range.

These above doses may be administered in a variety of volumes of carrier, excipient or buffer formulation, ranging from about 25 to about 1000 microliters, including all numbers within the range, depending on the size of the area to be treated, the viral titer used, the route of administration, and the desired effect of the method. In one embodiment, the volume of carrier, excipient or buffer is at least about 25 μL. In one embodiment, the volume is about 50 μL. In another embodiment, the volume is about 75 μL. In another embodiment, the volume is about 100 μL. In another embodiment, the volume is about 125 μL. In another embodiment, the volume is about 150 μL. In another embodiment, the volume is about 175 μL. In yet another embodiment, the volume is about 200 μL. In another embodiment, the volume is about 225 μL. In yet another embodiment, the volume is about 250 μL. In yet another embodiment, the volume is about 275 μL. In yet another embodiment, the volume is about 300 μL. In yet another embodiment, the volume is about 325 μL. In another embodiment, the volume is about 350 μL. In another embodiment, the volume is about 375 μL. In another embodiment, the volume is about 400 μL. In another embodiment, the volume is about 450 μL. In another embodiment, the volume is about 500 μL. In another embodiment, the volume is about 550 μL. In another embodiment, the volume is about 600 μL. In another embodiment, the volume is about 650 μL. In another embodiment, the volume is about 700 μL. In another embodiment, the volume is between about 700 and 1000 μL.

In other embodiments, volumes of about 14 to 150 mL may be selected, with the higher volumes being selected for adults. Typically, for newborn infants a suitable volume is about 0.5 mL to about 10 mL, for older infants, about 0.5 mL to about 15 mL may be selected. For toddlers, a volume of about 0.5 mL to about 20 mL may be selected. For children, volumes of up to about 30 mL may be selected. For pre-teens and teens, volumes up to about 50 mL may be selected. In still other embodiments, a patient may receive an intrathecal administration in a volume of about 5 mL to about 15 mL are selected, or about 7.5 mL to about 10 mL. Other suitable volumes and dosages may be determined. The dosage will be adjusted to balance the therapeutic benefit against any side effects and such dosages may vary depending upon the therapeutic application for which the recombinant vector is employed.

In one embodiment, the viral constructs may be delivered in doses of from at least 1×10⁹ to about least 1×10¹¹ GCs in volumes of about 1 μL to about 3 μL for small animal subjects, such as mice. For larger veterinary subjects, the larger human dosages and volumes stated above are useful. See, e.g., Diehl et al, J. Applied Toxicology, 21:15-23 (2001) for a discussion of good practices for administration of substances to various veterinary animals. This document is incorporated herein by reference.

The above-described recombinant vectors may be delivered to host cells according to published methods. The rAAV, preferably suspended in a physiologically compatible carrier, may be administered to a human or non-human mammalian patient. In another embodiment, the composition includes a carrier, diluent, excipient and/or adjuvant. Suitable carriers may be readily selected by one of skill in the art in view of the indication for which the transfer virus is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The buffer/carrier should include a component that prevents the rAAV, from sticking to the infusion tubing but does not interfere with the rAAV binding activity in vivo.

Optionally, the compositions of the invention may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The compositions according to the present invention may comprise a pharmaceutically acceptable carrier, such as defined above. Suitably, the compositions described herein comprise an effective amount of one or more AAV suspended in a pharmaceutically suitable carrier and/or admixed with suitable excipients designed for delivery to the subject via injection, osmotic pump, intrathecal catheter, or for delivery by another device or route. In one example, the composition is formulated for intrathecal delivery. In one embodiment, intrathecal delivery encompasses an injection into the spinal canal, e.g., the subarachnoid space.

The viral vectors described herein may be used in preparing a medicament for delivering hSMN1 to a subject (e.g., a human patient) in need thereof, supplying functional SMN to a subject, and/or for treating spinal muscular atrophy. A course of treatment may optionally involve repeat administration of the same viral vector (e.g., an AAVrh.10 vector) or a different viral vector (e.g., an AAV9 and an AAVrh10). Still other combinations may be selected using the viral vectors and non-viral delivery systems described herein.

The hSMN1 cDNA sequences described herein can be generated in vitro and synthetically, using techniques well known in the art. For example, the PCR-based accurate synthesis (PAS) of long DNA sequence method may be utilized, as described by Xiong et al, PCR-based accurate synthesis of long DNA sequences, Nature Protocols 1, 791-797 (2006). A method combining the dual asymmetrical PCR and overlap extension PCR methods is described by Young and Dong, Two-step total gene synthesis method, Nucleic Acids Res. 2004; 32(7): e59. See also, Gordeeva et al, J Microbiol Methods. Improved PCR-based gene synthesis method and its application to the Citrobacter freundii phytase gene codon modification. 2010 May; 81(2):147-52. Epub 2010 Mar. 10; see, also, the following patents on oligonucleotide synthesis and gene synthesis, Gene Seq. 2012 April; 6(1):10-21; U.S. Pat. No. 8,008,005; and U.S. Pat. No. 7,985,565. Each of these documents is incorporated herein by reference. In addition, kits and protocols for generating DNA via PCR are available commercially. These include the use of polymerases including, without limitation, Taq polymerase; OneTaq® (New England Biolabs); Q5® High-Fidelity DNA Polymerase (New England Biolabs); and GoTaq® G2 Polymerase (Promega). DNA may also be generated from cells transfected with plasmids containing the hSMN sequences described herein. Kits and protocols are known and commercially available and include, without limitation, QIAGEN plasmid kits; Chargeswitch® Pro Filter Plasmid Kits (Invitrogen); and GenElute™ Plasmid Kits (Sigma Aldrich). Other techniques useful herein include sequence-specific isothermal amplification methods that eliminate the need for thermocycling. Instead of heat, these methods typically employ a strand-displacing DNA polymerase, like Bst DNA Polymerase, Large Fragment (New England Biolabs), to separate duplex DNA. DNA may also be generated from RNA molecules through amplification via the use of Reverse Transcriptases (RT), which are RNA-dependent DNA Polymerases. RTs polymerize a strand of DNA that is complimentary to the original RNA template and is referred to as cDNA. This cDNA can then be further amplified through PCR or isothermal methods as outlined above. Custom DNA can also be generated commercially from companies including, without limitation, GenScript; GENEWIZ®; GeneArt® (Life Technologies); and Integrated DNA Technologies.

The term “expression” is used herein in its broadest meaning and comprises the production of RNA or of RNA and protein. With respect to RNA, the term “expression” or “translation” relates in particular to the production of peptides or proteins. Expression may be transient or may be stable.

The term “translation” in the context of the present invention relates to a process at the ribosome, wherein an mRNA strand controls the assembly of an amino acid sequence to generate a protein or a peptide.

According to the present invention, a “therapeutically effective amount” of the hSMN1 is delivered as described herein to achieve a desired result, i.e., treatment of SMA or one or more symptoms thereof. As described herein, a desired result includes reducing muscle weakness, increasing muscle strength and tone, preventing or reducing scoliosis, or maintaining or increasing respiratory health, or reducing tremors or twitching. Other desired endpoints can be determined by a physician.

In some instances, SMA is detected in a fetus at around 30 to 36 weeks of pregnancy. In this situation, it may be desirable to treat the neonate as soon as possible after delivery. It also may be desirable to treat the fetus in utero. Thus, a method of rescuing and/or treating a neonatal subject having SMA is provided, comprising the step of delivering a hSNM1 gene to the neuronal cells of a newborn subject (e.g., a human patient). A method of rescuing and/or treating a fetus having SMA is provided, comprising the step of delivering a hSMN1 gene to the neuronal cells of the fetus in utero. In one embodiment, the gene is delivered in a composition described herein via intrathecal injection. This method may utilize any nucleic acid sequence encoding a functional hSMN protein, whether a codon optimized hSMN1 as described herein or a native hSMN1, or an hSMN1 allele with potentiated activity, as compared to a “wild type” protein, or a combination thereof. In one embodiment, treatment in utero is defined as administering an hSMN1 construct as described herein after detection of SMA in the fetus. See, e.g., David et al, Recombinant adeno-associated virus-mediated in utero gene transfer gives therapeutic transgene expression in the sheep, Hum Gene Ther. 2011 April; 22(4):419-26. doi: 10.1089/hum.2010.007. Epub 2011 Feb. 2, which is incorporated herein by reference.

In one embodiment, neonatal treatment is defined as being administered an hSMN1 construct as described herein within 8 hours, the first 12 hours, the first 24 hours, or the first 48 hours of delivery. In another embodiment, particularly for a primate (human or non-human), neonatal delivery is within the period of about 12 hours to about 1 week, 2 weeks, 3 weeks, or about 1 month, or after about 24 hours to about 48 hours.

In another embodiment, for late onset SMA, the composition is delivered after onset of symptoms. In one embodiment, treatment of the patient (e.g., a first injection) is initiated prior to the first year of life. In another embodiment, treatment is initiated after the first 1 year, or after the first 2 to 3 years of age, after 5 years of age, after 11 years of age, or at an older age.

In another embodiment, the construct is readministered at a later date. Optionally, more than one readministration is permitted. Such readministration may be with the same type of vector, a different viral vector, or via non-viral delivery as described herein. For example, in the event a patient was treated with rAAV9 encoding SMN and requires a second treatment, rAAVrh.10.SMN can be subsequently administered, and vice-versa. Also, if a patient has neutralizing antibodies to AAV9, rAAVrh.10.SMN can be administered to the patient instead.

Treatment of SMA patients may require a combination therapy, such as transient co-treatment with an immunosuppressant before, during and/or after treatment with the compositions of the invention. Immunosuppressants for such co-therapy include, but are not limited to, steroids, antimetabolites, T-cell inhibitors, and alkylating agents. For example, such transient treatment may include a steroid (e.g., prednisole) dosed once daily for 7 days at a decreasing dose, in an amount starting at about 60 mg, and decreasing by 10 mg/day (day 7 no dose). Other doses and immunosuppressants may be selected.

By “functional hSMN1”, is meant a gene which encodes the native SMN protein such as that characterized by SEQ ID NO: 1 or another SMN protein which provides at least about 50%, at least about 75%, at least about 80%, at least about 90%, or about the same, or greater than 100% of the biological activity level of the native survival of motor neuron protein, or a natural variant or polymorph thereof which is not associated with disease. Additionally, SMN1homologue-SMN2 also encodes the SMN protein, but processes the functional protein less efficiently. Based on the copy number of SMN2, subjects lacking a functional hSMN1 gene demonstrate SMA to varying degrees. Thus, for some subjects, it may be desirable for the SMN protein to provide less than 100% of the biological activity of the native SMN protein.

In one embodiment, such a functional SMN has a sequence which has about 95% or greater identity to the native protein, or full-length sequence of SEQ ID NO: 1, or about 97% identity or greater, or about 99% or greater to SEQ ID NO: 1 at the amino acid level. Such a functional SMN protein may also encompass natural polymorphs. Identity may be determined by preparing an alignment of the sequences and through the use of a variety of algorithms and/or computer programs known in the art or commercially available [e.g., BLAST, ExPASy; ClustalO; FASTA; using, e.g., Needleman-Wunsch algorithm, Smith-Waterman algorithm].

A variety of assays exist for measuring SMN expression and activity levels in vitro. See, e.g., Tanguy et al, 2015, cited above. The methods described herein can also be combined with any other therapy for treatment of SMA or the symptoms thereof. See, also, Wang et al, Consensus Statement for Standard of Care in Spinal Muscular Atropy, which provides a discussion of the present standard of care for SMA and http://www.ncbi.nlm.nih.gov/books/NBK1352/. For example, when nutrition is a concern in SMA, placement of a gastrostomy tube is appropriate. As respiratory function deteriorates, tracheotomy or noninvasive respiratory support is offered. Sleep-disordered breathing can be treated with nighttime use of continuous positive airway pressure. Surgery for scoliosis in individuals with SMA II and SMA III can be carried out safely if the forced vital capacity is greater than 30%-40%. A power chair and other equipment may improve quality of life. See also, U.S. Pat. No. 8,211,631, which is incorporated herein by reference.

It is to be noted that the term “a” or “an” refers to one or more. As such, the terms “a” (or “an”), “one or more,” and “at least one” are used interchangeably herein.

The words “comprise”, “comprises”, and “comprising” are to be interpreted inclusively rather than exclusively. The words “consist”, “consisting”, and its variants, are to be interpreted exclusively, rather than inclusively. While various embodiments in the specification are presented using “comprising” language, under other circumstances, a related embodiment is also intended to be interpreted and described using “consisting of” or “consisting essentially of” language.

As used herein, the term “about” means a variability of 10% (±10%) from the reference given, unless otherwise specified.

As used herein, “disease”, “disorder” and “condition” are used interchangeably, to indicate an abnormal state in a subject.

Unless defined otherwise in this specification, technical and scientific terms used herein have the same meaning as commonly understood by one of ordinary skill in the art and by reference to published texts, which provide one skilled in the art with a general guide to many of the terms used in the present application.

The following examples are illustrative only and are not intended to limit the present invention.

### Example 1—AAV Vectors Containing hSMN1

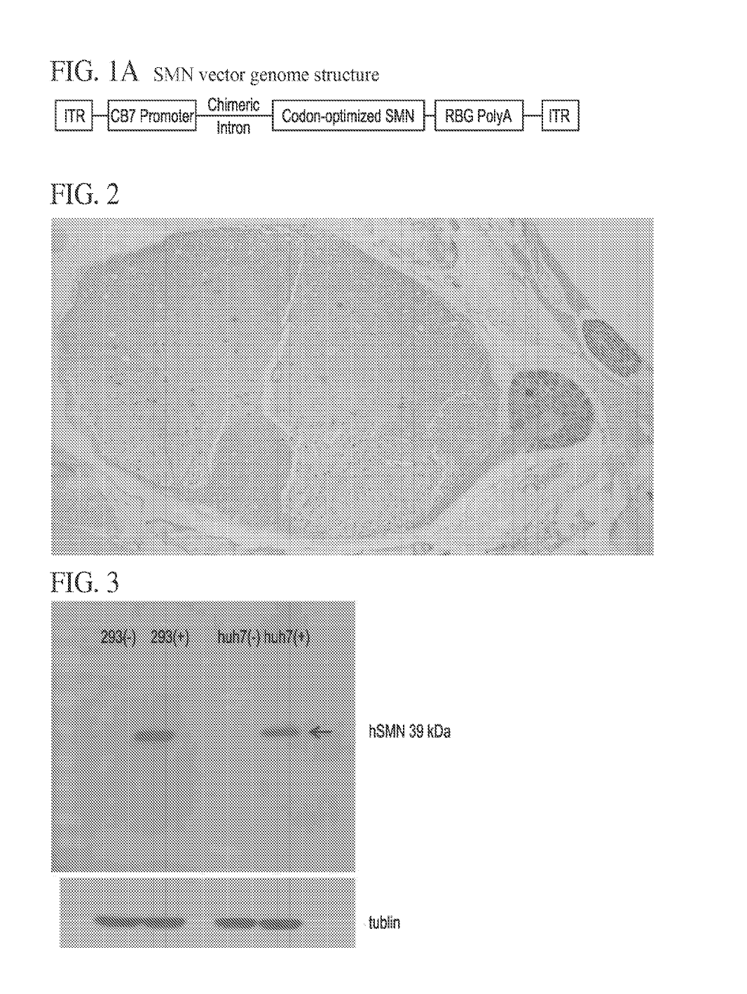

Using the SMNΔ7 mouse model, we evaluated AAV-mediated gene therapy for the treatment of SMA. A neurotropic AAVrh.10 vector was constructed bearing a codon-optimized human SMN1 cDNA under the control of a ubiquitous CB promoter (FIG. 1). Newborn SMNΔ7 pups were injected with 5×10¹⁰ genome copies of the vector (5×10¹³ genome copies/kg) via the facial vein. Treatment resulted in robust expression in peripheral neurons such as dorsal root ganglia (FIG. 2), as well as transduction within the spinal cord at this dose. Some improvement in survival (21 days vs 14 in untreated mice) was also observed.

### Example 2—Additional Dosage Studies

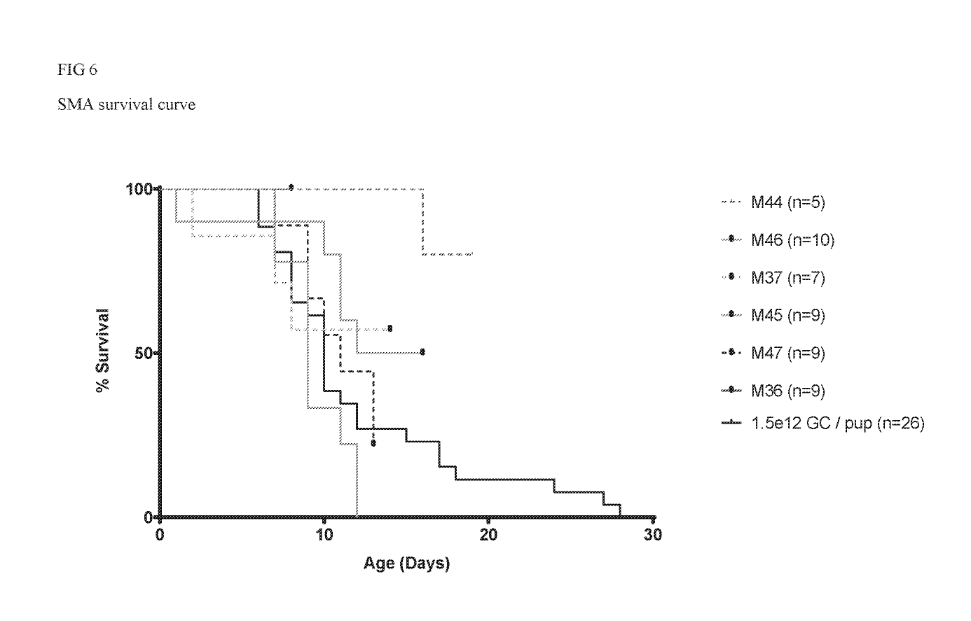

Newborn SMNΔ7 pups were injected with 5×10¹² genome copies/pup of the vector via IV injection. The median survival of the pups was 10 days. Aspartate aminotransferase (AST) and Alanine aminotransferase (ALT) levels were elevated. FIG. 6.

49 SMNΔ7 pups in the age range of 4-15 days were injected with 5×10¹¹ genome copies/pup of the vector via IV injection. The designations M44, M46, M37, M45, M47 and M36 refer to the different litters of pups used in the study. At day 30, 49 pups remained alive. FIG. 6.

### Example 3: Intrathecal Delivery of AAV Vectors Containing hSMN

The dosing and efficacy of AAVrh.10.SMN delivered directly to the cerebral spinal fluid (CSF) via single injection is evaluated.

Intracerebroventricular (ICV) delivery of AAVrh.10.SMN or sAAVrh.10.GFP is evaluated in newborn SMNΔ7 pups. Animals from each treatment group are sacrificed at 7, 14, 30, 60 or 90 days after vector administration for analysis of vector biodistribution and enzyme expression. Mice are monitored daily of survival and weight gain. Behavioral testing on the mice includes being tested for righting reflex by determining their ability to right themselves within 30 seconds after being put on their side. The dose of AAVrh.10.SMN that rescues the phenotype of the pups is determined and is informative as to the dose administered to the pig SMA model.

Intrathecal delivery of AAVrh.10.SMN or sAAVrh.10.GFP is evaluated in a pig SMA model, as described in Duque et al. Ann Neurol. 2015, 77(3): 399-414. Longitudinal electrophysiological studies, histology, and neuropathology studies are performed for analysis of efficacy, vector biodistribution, and enzyme expression. The dose of AAVrh.10.SMN that rescues the phenotype of the pigs is determined and is informative as to the dose for administered to non-human primates and humans.

Cynomolus macaques are administered sAAVrh.10.GFP using a single intrathecal sacral infusion or injection. Two weeks following dosing, the macaques are euthanized and immunofluorescence staining is performed for analysis of vector biodistribution and enzyme expression and DNA and RNA biodistribution.

### Sequence Listing Free Text

The following information is provided for sequences containing free text under numeric identifier <223>.

| SEQ ID NO: | |
|---|---|
| (containing free text) | Free text under <223> |
| 2 | <223> constructed sequence |
| 4 | <223> constructed sequence |
| 5 | <223> Adeno-associated virus |
| | rh10 VP1 protein |

All published documents cited in this specification and priority document U.S. Provisional Patent Application No. 62/267,012, filed Dec. 14, 2014, are incorporated herein by reference in their entirety. Similarly, the SEQ ID NO which are referenced herein and which appear in the appended Sequence Listing are incorporated by reference. While the invention has been described with reference to particular embodiments, it will be appreciated that modifications can be made without departing from the spirit of the invention. Such modifications are intended to fall within the scope of the appended claims.

1. A recombinant adeno-associated viral (AAV) vector comprising an AAVrh10 capsid and a vector genome comprising a nucleic acid sequence encoding a functional SMN protein and expression control sequences that direct expression of the SMN sequences in a host cell.

2. The AAV vector of claim 1, wherein the AAV capsid is an AAVrh.10 capsid comprising an amino acid sequence of SEQ ID NO: 5 or a sequence at least about 99% identical thereto.

3. The AAV vector of claim 1, wherein the nucleic acid sequences encode SEQ ID NO: 1 or a sequence sharing 95% identity therewith.

4. The AAV vector of claim 1, wherein the nucleic acid sequence encoding SMN is the SMN1 sequence of SEQ ID NO: 2, or a sequence sharing at least 70% identity therewith.

5. The AAV vector according to claim 4, wherein the sequence sharing at least 70% identity with SEQ ID NO: 2 is a codon optimized sequence.

6. The AAV vector of claim 1, wherein the expression control sequences comprise a promoter.

7. (canceled)

8. The AAV vector of claim 6, wherein the promoter is a CB7 promoter.

9. (canceled)

10. The AAV vector of claim 6, wherein the promoter is a neuron-specific promoter.

11. The AAV vector of claim 1, further comprising one or more of an intron, a Kozak sequence, a polyA, WPRE, and post-transcriptional regulatory elements.

12. The AAV vector of claim 1, further comprising AAV inverted terminal repeat (ITRs) sequences.

13. The viral vector of claim 12, wherein the ITRs are from an AAV different from the AAV supplying the capsid.

14. The viral vector of claim 12, wherein the ITRs are from AAV2.

15. A pharmaceutical composition comprising a pharmaceutically acceptable carrier and a viral vector according to claim 1.

16-21. (canceled)

22. A method for treating spinal muscular atrophy in a subject, said method comprising administering the composition of claim 15 to a subject in need thereof.

23. The method according to claim 22, wherein said composition is administered intrathecally.

24. The method according to claim 22, wherein said subject is a mammal.

25. The method according to claim 22, wherein said subject is a human.

26. The method according to claim 22, wherein said composition is administered in combination with another therapy.

27. The method according to claim 22, wherein said vector is administered at a dosage of from about 1×10¹⁰ GC/kg to about 1×10¹⁴ GC/kg.

28. The method according to claim 22, wherein said vector or composition is administered more than once.


SEQUENCE LISTING (5 sequences)

SEQ ID NO: 1 - protein, 294 aa, Homo sapiens
MAMSSGGSGGGVPEQEDSVLFRRGTGQSDDSDIWDDTALIKAYDKAVASFKHALKNGDICETSGKPKTTPKRKPAKKNKSQKKNTAASLQQWKVGDKCSAIWSEDGCIYPATIASIDFKRETCVVVYTGYGNREEQNLSDLLSPICEVANNIEQNAQENENESQVSTDESENSRSPGNKSDNIKPKSAPWNSFLPPPPPMPGPRLGPGKPGLKFNGPPPPPPPPPPHLLSCWLPPFPSGPPIIPPPPPICPDSLDDADALGSMLISWYMSGYHTGYYMGFRQNQKEGRCSHSLN

SEQ ID NO: 2 - DNA, 882 bp, Artificial Sequence
  constructed sequence
atggccatgtcgagtgggggcagtggagggggagtgccagaacaggaagattccgtgctgttcaggcgaggaaccgggcagagtgacgacagtgacatttgggacgacacggccctgatcaaggcctatgacaaagccgtggcctccttcaagcacgcgctgaagaacggcgacatttgcgaaaccagcggcaagcctaagaccacccctaaacggaagcccgccaagaaaaataagtcccagaaaaagaacacagccgcaagtcttcagcaatggaaggtgggggataagtgctccgcgatatggagtgaagacgggtgcatctatcctgccaccatcgccagcatagacttcaagcgcgaaacctgcgtggtggtgtacactggatacgggaaccgggaggagcagaacctgagcgacctgttgagccctatttgtgaggtggccaacaacatcgagcagaatgcgcaagaaaatgaaaacgagagtcaggtgtccaccgatgagagtgaaaacagtaggagccccggcaacaaatccgacaatatcaagcccaaaagcgcaccctggaatagcttccttccacccccccccccaatgcccggacctcgactgggccccggaaagcctggcctgaagttcaacggcccccctcctcctcctccccctcctcccccccacctgctgagctgctggttgccccctttcccttcgggaccccctatcatacctccccccccccctatttgccctgactccctggacgacgcggacgcgctgggcagtatgctcatctcgtggtacatgtcaggataccacaccgggtactacatgggcttcagacaaaatcagaaggaaggacgatgtagtcactccctgaat

SEQ ID NO: 3 - DNA, 885 bp, Homo sapiens
atggcgatgagcagcggcggcagtggtggcggcgtcccggagcaggaggattccgtgctgttccggcgcggcacaggccagagcgatgattctgacatttgggatgatacagcactgataaaagcatatgataaagctgtggcttcatttaagcatgctctaaagaatggtgacatttgtgaaacttcgggtaaaccaaaaaccacacctaaaagaaaacctgctaagaagaataaaagccaaaagaagaatactgcagcttccttacaacagtggaaagttggggacaaatgttctgccatttggtcagaagacggttgcatttacccagctaccattgcttcaattgattttaagagagaaacctgtgttgtggtttacactggatatggaaatagagaggagcaaaatctgtccgatctactttccccaatctgtgaagtagctaataatatagaacaaaatgctcaagagaatgaaaatgaaagccaagtttcaacagatgaaagtgagaactccaggtctcctggaaataaatcagataacatcaagcccaaatctgctccatggaactcttttctccctccaccaccccccatgccagggccaagactgggaccaggaaagccaggtctaaaattcaatggcccaccaccgccaccgccaccaccaccaccccacttactatcatgctggctgcctccatttccttctggaccaccaataattcccccaccacctcccatatgtccagattctcttgatgatgctgatgctttgggaagtatgttaatttcatggtacatgagtggctatcatactggctattatatgggtttcagacaaaatcaaaaagaaggaaggtgctcacattccttaaattaa

SEQ ID NO: 4 - DNA, 6079 bp, Artificial Sequence
  constructed sequence
aattctagcttgccaccatggccatgtcgagtgggggcagtggagggggagtgccagaacaggaagattccgtgctgttcaggcgaggaaccgggcagagtgacgacagtgacatttgggacgacacggccctgatcaaggcctatgacaaagccgtggcctccttcaagcacgcgctgaagaacggcgacatttgcgaaaccagcggcaagcctaagaccacccctaaacggaagcccgccaagaaaaataagtcccagaaaaagaacacagccgcaagtcttcagcaatggaaggtgggggataagtgctccgcgatatggagtgaagacgggtgcatctatcctgccaccatcgccagcatagacttcaagcgcgaaacctgcgtggtggtgtacactggatacgggaaccgggaggagcagaacctgagcgacctgttgagccctatttgtgaggtggccaacaacatcgagcagaatgcgcaagaaaatgaaaacgagagtcaggtgtccaccgatgagagtgaaaacagtaggagccccggcaacaaatccgacaatatcaagcccaaaagcgcaccctggaatagcttccttccacccccccccccaatgcccggacctcgactgggccccggaaagcctggcctgaagttcaacggcccccctcctcctcctccccctcctcccccccacctgctgagctgctggttgccccctttcccttcgggaccccctatcatacctccccccccccctatttgccctgactccctggacgacgcggacgcgctgggcagtatgctcatctcgtggtacatgtcaggataccacaccgggtactacatgggcttcagacaaaatcagaaggaaggacgatgtagtcactccctgaattaatgatagctagaattcacgcgtggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaag

SEQ ID NO: 5 - protein, 738 aa, Artificial sequence
  Adeno-associated virus rh10 VP1 protein
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL